(19) United States
(12) Patent Application Publication
(10) Pub. No.: US 2026/0137536 A1
(43) Pub. Date: May 21, 2026

(54) STENTS, STENT DELIVERY SYSTEMS, AND METHODS

(71) Applicant: BOSTON SCIENTIFIC SCIMED, INC., Maple Grove, MN (US)

(72) Inventors: Cristina Romany, Newton, MA (US); Jasmine Bourgerie, Sherborn, MA (US); Kurt Nicholas Robakiewicz, Waltham, MA (US); Rahul Prabhu, Brighton, MA (US)

(73) Assignee: BOSTON SCIENTIFIC SCIMED, INC., Maple Grove, MN (US)

(21) Appl. No.: 19/393,103

(22) Filed: Nov. 18, 2025

Related U.S. Application Data
(60) Provisional application No. 63/722,363, filed on Nov. 19, 2024.

(51) Int. Cl. A61F 2/958; A61F 2/966

(52) U.S. Cl. CPC A61F 2/958; A61F 2/966

(57) ABSTRACT

A stent delivery system includes a delivery sheath having a distal expandable region, and an elongate shaft disposed within the sheath, the shaft including a dilation balloon at its distal end. The expandable region is configured to constrain a self-expanding stent in a collapsed configuration. The balloon is configured to shift the stent to an expanded configuration within the distal expandable region. A method of delivering a self-expanding stent includes positioning the stent delivery system at a target site, inflating the balloon within the expandable region, retracting the sheath with the balloon in an inflated configuration to move the stent out of the expandable region, and thereafter, deflating the balloon to release the stent. A stent delivery system includes a delivery sheath configured to receive a stent in a collapsed configuration, and a balloon disposed within the sheath. The balloon is configured to advance the stent out of the sheath.

### CROSS REFERENCE TO RELATED APPLICATIONS

The present application claims the benefit of US Provisional Patent Application Serial No. 63/722,363, filed November 19, 2024, the disclosure of which is incorporated herein by reference.

### TECHNICAL FIELD

The disclosure relates generally to medical devices and more particularly to stents, stent delivery systems and components thereof, and associated methods.

### BACKGROUND

A wide variety of intracorporeal medical devices and systems have been developed for medical use, for example, surgical and/or intravascular use. Some of these devices include guidewires, catheters, stents, endoprostheses, medical device systems, and the like. A stent may be configured to be positioned in a body lumen for a variety of medical applications. For example, a stent may be used to treat a stenosis in a blood vessel, used to maintain a fluid opening or pathway in the vascular, urinary, biliary, tracheobronchial, esophageal, or renal tracts, or to position a device such as an artificial valve or filter within a body lumen. The known devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and/or using medical devices.

### SUMMARY

In one example, a stent delivery system may comprise a delivery sheath comprising a distal expandable region and a proximal non-expandable region, and an elongate shaft slidably disposed within the delivery sheath. The elongate shaft may comprise a dilation balloon at its distal end. The distal expandable region may be configured to constrain a self-expanding stent in a radially collapsed configuration with the dilation balloon disposed within the self-expanding stent in a deflated configuration. The dilation balloon may be configured to inflate to an inflated configuration within the distal expandable region to shift the self-expanding stent to a radially expanded configuration within the distal expandable region.

In addition, or alternatively, to any example disclosed herein, the distal expandable region of the delivery sheath is configured to expand radially outward to a deployment configuration upon inflation of the dilation balloon to the inflated configuration within the distal expandable region.

In addition, or alternatively, to any example disclosed herein, the delivery sheath is retractable relative to the elongate shaft while the self-expanding stent is in the radially expanded configuration to deploy the self-expanding stent.

In addition, or alternatively, to any example disclosed herein, the dilation balloon is configured to retain the self-expanding stent thereon in the inflated configuration as the delivery sheath is retracted relative to the elongate shaft.

In addition, or alternatively, to any example disclosed herein, the distal expandable region is configured to radially collapse toward a delivery configuration after deploying the self-expanding stent.

In addition, or alternatively, to any example disclosed herein, the distal expandable region is self-biased toward the delivery configuration.

In addition, or alternatively, to any example disclosed herein, a method of delivering a self-expanding stent may comprise: positioning a stent delivery system with the self-expanding stent disposed therein in a radially collapsed configuration at a target site, wherein the stent delivery system comprises: a delivery sheath comprising a distal expandable region and a proximal non-expandable region; and an elongate shaft slidably disposed within the delivery sheath, wherein the elongate shaft comprises a dilation balloon at its distal end; inflating the dilation balloon to an inflated configuration within the distal expandable region of the delivery sheath with the self-expanding stent disposed within the distal expandable region of the delivery sheath; retracting the delivery sheath relative to the elongate shaft with the dilation balloon in the inflated configuration to move the self-expanding stent out of the distal expandable region; and thereafter, deflating the dilation balloon to release the self-expanding stent at the target site.

In addition, or alternatively, to any example disclosed herein, the distal expandable region is configured to constrain the self-expanding stent in the radially collapsed configuration with the dilation balloon disposed within the self-expanding stent in a deflated configuration.

In addition, or alternatively, to any example disclosed herein, inflating the dilation balloon to an inflated configuration within the distal expandable region of the delivery sheath shifts the self-expanding stent to a radially expanded configuration within the distal expandable region.

In addition, or alternatively, to any example disclosed herein, inflating the dilation balloon to an inflated configuration within the distal expandable region of the delivery sheath radially expands the distal expandable region of the delivery sheath to a deployment configuration.

In addition, or alternatively, to any example disclosed herein, in the inflated configuration the dilation balloon is configured to retain the self-expanding stent thereon as the delivery sheath is retracted.

In addition, or alternatively, to any example disclosed herein, the method may comprise radially collapsing the distal expandable region of the delivery sheath toward a delivery configuration and removing the stent delivery system from the target site.

In addition, or alternatively, to any example disclosed herein, the distal expandable region of the delivery sheath is self-biased toward the delivery configuration.

In addition, or alternatively, to any example disclosed herein, a stent delivery system may comprise a delivery sheath configured to receive a stent within a distal portion of the delivery sheath in a radially collapsed configuration, and a balloon disposed within the distal portion of the delivery sheath. The balloon may be configured to advance the stent out of the distal portion of the delivery sheath.

In addition, or alternatively, to any example disclosed herein, the balloon is fixedly attached to the delivery sheath.

In addition, or alternatively, to any example disclosed herein, the balloon is configured to expand radially inward from the delivery sheath to advance the stent out of the distal portion of the delivery sheath.

In addition, or alternatively, to any example disclosed herein, the balloon is configured to constrain the stent in the radially collapsed configuration within the distal portion of the delivery sheath.

In addition, or alternatively, to any example disclosed herein, the balloon is fixedly attached to a distal end of an elongate shaft disposed within the delivery sheath.

In addition, or alternatively, to any example disclosed herein, the balloon is configured to expand distally from the distal end of the elongate shaft to advance the stent out of the distal portion of the delivery sheath.

In addition, or alternatively, to any example disclosed herein, the elongate shaft is configured to advance distally relative to the delivery sheath with the balloon in an inflated configuration to push the stent out of the distal portion of the delivery sheath.

Another example is a stent delivery system for delivering a self-expanding stent. The stent delivery system includes a delivery sheath comprising a distal expandable region and a proximal non-expandable region, and an elongate shaft slidably disposed within the delivery sheath. The elongate shaft comprises a dilation balloon at its distal end. A self-expanding stent surrounds the dilatation balloon in a radially collapsed configuration with the dilation balloon in a deflated configuration within a lumen of the distal expandable region of the delivery sheath in a non-expanded configuration. The dilation balloon is configured to be inflated to an inflated configuration within the distal expandable region of the delivery sheath with the self-expanding stent disposed within the distal expandable region of the delivery sheath to expand the self-expanding stent and the distal expandable region to a radially expanded configuration.

In addition, or alternatively, to any example disclosed herein, the distal expandable region is configured to constrain the self-expanding stent in the radially collapsed configuration with the dilation balloon disposed within the self-expanding stent in the deflated configuration.

In addition, or alternatively, to any example disclosed herein, the expandable region has a first outer diameter in the non-expanded configuration and the expandable region has a second outer diameter in the radially expanded configuration, the second outer diameter being greater than the first outer diameter.

In addition, or alternatively, to any example disclosed herein, the delivery sheath is retractable relative to the elongate shaft while the self-expanding stent is in the radially expanded configuration to deploy the self-expanding stent.

In addition, or alternatively, to any example disclosed herein, in the inflated configuration the dilation balloon is configured to retain the self-expanding stent thereon as the delivery sheath is retracted.

In addition, or alternatively, to any example disclosed herein, the distal expandable region of the delivery sheath is configured to radially collapse toward the non-expanded configuration when the self-expanding stent is deployed from the distal expandable region of the delivery sheath.

In addition, or alternatively, to any example disclosed herein, the distal expandable region of the delivery sheath is self-biased toward the non-expanded configuration.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-6 schematically illustrate selected aspects of a stent delivery system and methods of delivering a stent;

FIGS. 7-11 schematically illustrate selected aspects of a stent delivery system and methods of delivering a stent;

FIGS. 12-14 schematically illustrate selected aspects of a stent delivery system and methods of delivering a stent;

FIGS. 15-16 are end views schematically illustrating selected aspects of a stent and a stent delivery system; and

FIGS. 17-19 are end views schematically illustrating selected aspects of a stent.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the disclosure. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

In some embodiments, a stent according to the disclosure may comprise an expandable framework having a first end, a second end, and a lumen extending therethrough. In some embodiments, the expandable framework may be and or define a generally tubular structure. The stent and/or the expandable framework may include a central longitudinal axis extending from the first end to the second end. The stent and/or the expandable framework may be configured to expand and/or self-expand from a radially collapsed configuration to a radially expanded configuration. In some embodiments, the stent and/or the expandable framework may be configured to self-expand from the radially collapsed configuration to the radially expanded configuration when unconstrained. In some embodiments, the stent and/or the expandable framework may be balloon expandable. In some embodiments, the stent and/or the expandable framework may be mechanically expandable. In some embodiments, the stent and/or the expandable framework in the radially collapsed configuration may be elongated and/or longer than in the radially expanded configuration. As such, in some embodiments, when the stent and/or the expandable framework is radially expanded, the stent and/or the expandable framework may shorten axially (e.g., foreshorten). In the interest of brevity, the disclosure is made in the context of a self-expanding stent, and the terms “stent” and “self-expanding stent” may be used interchangeably herein. However, the skilled person shall recognize that the stent does not necessarily have to be a self-expanding stent, and in some embodiments, non-self-expanding stents may be used with the stent delivery systems and methods disclosed herein.

In some embodiments, the stent and/or the expandable framework may include a first flared region proximate the first end and a second flared region proximate the second end. The stent and/or the expandable framework may include a body region. In some embodiments, the body region may extend from the first flared region to the second flared region. In some embodiments, the first flared region and/or the second flared region have an outer diameter in the radially expanded configuration, wherein an outer diameter of the body region is less than the outer diameter of the first flared region and/or the second flared region.

In some embodiments, the stent and/or the expandable framework may include a plurality of interwoven filaments, such as when forming a braided stent. In some embodiments, the stent and/or the expandable framework may be a monolithic structure including a plurality of interconnected struts and/or a single filament interwoven with itself, such as when forming a knitted stent. In some embodiments, the stent and/or the expandable framework may be cut from a tubular member, using various means known in the art (e.g., laser, machining, etching, chemical dissolution, etc.). Other types and/or forms of stents are also contemplated. In one preferred configuration, the stent and/or the expandable framework may be formed from a metallic material. In some embodiments, the stent and/or the expandable framework may be formed from a polymeric material, a composite material, combinations thereof, etc. Some examples of suitable but non-limiting materials for the stent and/or the expandable framework, and/or elements or components thereof, are described below.

In some embodiments, the stent may be a covered stent. In some embodiments, the stent may include a polymeric covering disposed on and/or disposed radially outward of an outer surface of the expandable framework. In some embodiments, the polymeric covering may be disposed on and/or disposed radially inward of an inner surface of the expandable framework. In some embodiments, the expandable framework may be embedded within the polymeric covering. In some embodiments, the polymeric covering may be impervious to fluids, debris, and/or tissue ingrowth. In some alternative embodiments, the polymeric covering may be configured to promote tissue ingrowth. Other configurations are also contemplated.

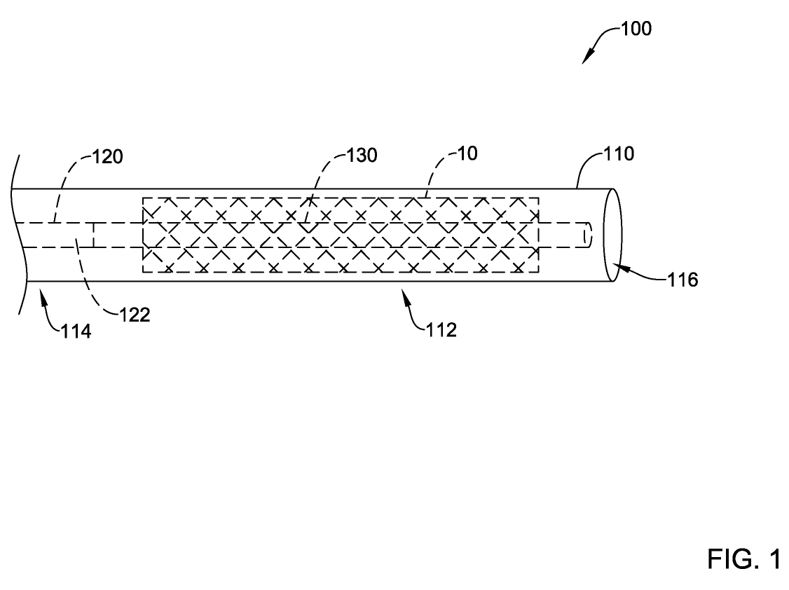

FIGS. 1-6 schematically illustrate selected aspects of a stent delivery system 100 and methods of delivering a self-expanding stent 10. The self-expanding stent 10 may be constructed and/or may comprise characteristics according to the instant disclosure. As discussed above, in some alternative embodiments, the stent may be a non-self-expanding stent.

In some embodiments, the stent delivery system 100 may comprise a delivery sheath 110 comprising a distal expandable region 112 and a proximal non-expandable region 114. The delivery sheath 110 may comprise a lumen 116 extending therethrough. The self-expanding stent 10 may be disposable within the distal expandable region 112 of the delivery sheath 110 in a radially collapsed configuration.

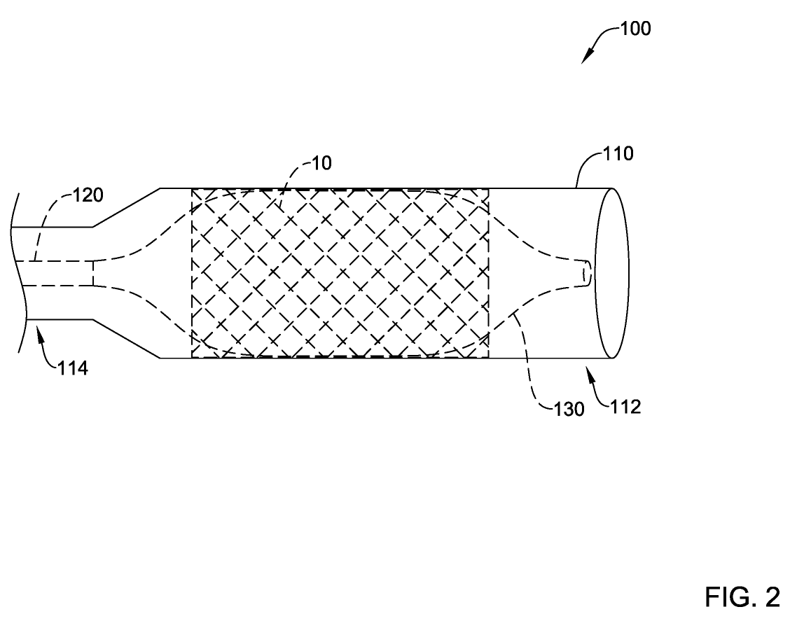

In some embodiments, the distal expandable region 112 may have a first outer diameter and/or a first outermost radial extent in a delivery configuration (e.g., FIG. 1), and a second outer diameter and/or a second outermost radial extent in a deployment configuration (e.g., FIG. 2), wherein the second outer diameter and/or the second outermost radial extent is greater than the first outer diameter and/or the first outermost radial extent. In some embodiments, the proximal non-expandable region 114 may have a third outer diameter and/or a third outermost radial extent. The third outer diameter and/or the third outermost radial extent is less than the second outer diameter and/or the second outermost radial extent. In some embodiments, the third outer diameter and/or the third outermost radial extent may be substantially equal to the first outer diameter and/or the first outermost radial extent. In some embodiments, the third outer diameter and/or the third outermost radial extent may be less than the first outer diameter and/or the first outermost radial extent. Other configurations are also contemplated.

The stent delivery system 100 may comprise an elongate shaft 120 slidably disposed within the delivery sheath 110 and/or the lumen 116 of the delivery sheath 110. The elongate shaft 120 may comprise a dilation balloon 130 at its distal end. In at least some embodiments, the dilation balloon 130 may be at a distalmost end of the elongate shaft 120 and/or may be a distalmost feature of the elongate shaft 120. The dilation balloon 130 may be configured to shift between a deflated configuration and an inflated configuration. The elongate shaft 120 may comprise an inflation lumen 122 disposed and/or extending therein. The elongate shaft 120 and/or the inflation lumen 122 may be in fluid communication with a source of inflation media (e.g., fluid, gas, etc.). The elongate shaft 120 and/or the dilation balloon 130 may be configured to translate relative to the delivery sheath 110. In some embodiments, the dilation balloon 130, in the deflated configuration, may be disposable within the self-expanding stent 10 in the radially collapsed configuration. In some embodiments, the dilation balloon 130, in the deflated configuration, may be configured to be disposed within the self-expanding stent 10 in the radially collapsed configuration.

In some embodiments, the dilation balloon 130 may be configured to radially expand the self-expanding stent 10 and/or the distal expandable region 112 of the delivery sheath 110 when shifting from the deflated configuration to the inflated configuration. In some embodiments, the dilation balloon 130 may be a compliant balloon. In some embodiments, the dilation balloon 130 may be a non-compliant balloon or a semi-compliant balloon. In at least some embodiments, the dilation balloon 130 may be formed from a polymeric material. Other configurations and/or materials are also contemplated.

In some embodiments, the distal expandable region 112 of the delivery sheath 110 may be configured to constrain the self-expanding stent 10 in the radially collapsed configuration when the distal expandable region 112 is in the delivery configuration. In some embodiments, the distal expandable region 112 of the delivery sheath 110 may have properties and/or characteristics capable of maintaining the self-expanding stent 10 in the radially collapsed configuration when the distal expandable region 112 is in the delivery configuration. In some embodiments, the distal expandable region 112 of the delivery sheath 110 may have properties and/or characteristics capable of resisting and/or overcoming a radial expansion force exerted on the distal expandable region 112 of the delivery sheath 110 by the self-expanding stent 10, thereby preventing the self-expanding stent 10 from shifting out of the radially collapsed configuration and/or toward a radially expanded configuration when the distal expandable region 112 is in the delivery configuration. In some embodiments, the radial expansion force exerted on the distal expandable region 112 of the delivery sheath 110 by the self-expanding stent 10 may be between about 0.25 Newtons (N) and about 5 N. Other configurations are also contemplated.

In some embodiments, the distal expandable region 112 of the delivery sheath 110 may be configured to constrain the self-expanding stent 10 in the radially collapsed configuration with the dilation balloon 130 disposed within the self-expanding stent 10 in the deflated configuration when the distal expandable region 112 is in the delivery configuration, as seen in FIG. 1. In some embodiments, the dilation balloon 130 may be configured to inflate to the inflated configuration within the distal expandable region 112 of the delivery sheath 110. In some embodiments, the dilation balloon 130 may be configured to inflate to the inflated configuration within the distal expandable region 112 of the delivery sheath 110 to shift the self-expanding stent 10 to the radially expanded configuration within the distal expandable region 112 of the delivery sheath 110, as seen in FIG. 2. In some embodiments, the distal expandable region 112 of the delivery sheath 110 may be configured to expand radially outward to the deployment configuration upon inflation of the dilation balloon 130 to the inflated configuration within the distal expandable region 112 of the delivery sheath 110. In some embodiments, inflation of the dilation balloon 130 within the self-expanding stent 10 disposed within the distal expandable region 112 of the delivery sheath 110 may be configured to simultaneously shift the self-expanding stent 10 to the radially expanded configuration and shift the distal expandable region 112 of the delivery sheath 110 to the deployment configuration. Other configurations are also contemplated.

In some embodiments, the delivery sheath 110 may be configured to be translatable relative to the elongate shaft 120 and/or the dilation balloon 130 while the self-expanding stent 10 is in the radially expanded configuration to deploy the self-expanding stent 10 at a target site. In some embodiments, the delivery sheath 110 may be retractable proximally relative to the elongate shaft 120 and/or the dilation balloon 130 while the self-expanding stent 10 is in the radially expanded configuration to deploy the self-expanding stent 10 at the target site. In some embodiments, the delivery sheath 110 may be retractable proximally relative to the elongate shaft 120 and/or the dilation balloon 130 while the dilation balloon 130 is in the inflated configuration to deploy the self-expanding stent 10 at the target site. In some embodiments, the delivery sheath 110 may be retractable proximally relative to the elongate shaft 120 and/or the dilation balloon 130 while the dilation balloon 130 is in the inflated configuration and/or the self-expanding stent 10 is in the radially expanded configuration to deploy the self-expanding stent 10 at the target site. In some alternative embodiments, the elongate shaft 120 and/or the dilation balloon 130 may be advanceable distally relative to the delivery sheath 110 while the dilation balloon 130 is in the inflated configuration and/or the self-expanding stent 10 is in the radially expanded configuration to deploy the self-expanding stent 10 at the target site.

In at least some embodiments, the delivery sheath 110 and/or the distal expandable region 112 may comprise a lubricious coating disposed on an outer surface thereof. In some embodiments, the lubricious coating may be configuration to ease translation of the delivery sheath 110 and/or the distal expandable region 112 relative to surrounding anatomy at the target site. In some embodiments, the delivery sheath 110 and/or the distal expandable region 112 may comprise and/or may be formed from a lubricious material. Other configurations, including combinations thereof, are also contemplated.

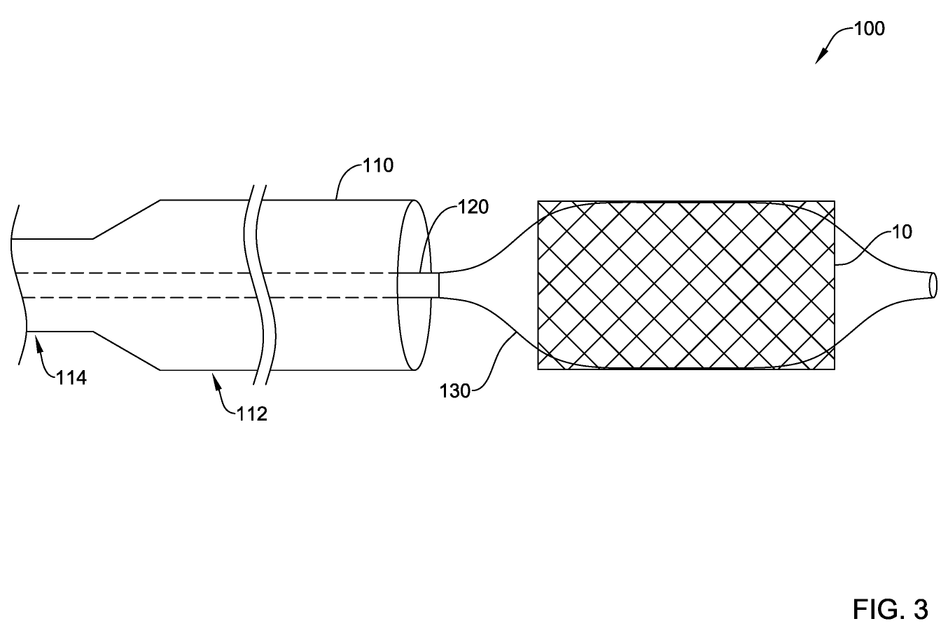

In some embodiments, the dilation balloon 130 may be configured to retain the self-expanding stent 10 thereon in the inflated configuration as the delivery sheath 110 is translated and/or retracted relative to the elongate shaft 120 and/or the dilation balloon 130 to deploy the self-expanding stent 10 at the target site, as seen in FIG. 3. In some embodiments, an outer surface of the dilation balloon 130 may be configured to engage with an inner surface of the self-expanding stent 10 in the inflated configuration to frictionally retain the self-expanding stent 10 thereon as the delivery sheath 110 is translated and/or retracted relative to the elongate shaft 120 and/or the dilation balloon 130 to deploy the self-expanding stent 10 at the target site. Other configurations and/or means of retaining the self-expanding stent 10 on the dilation balloon 130 in the inflated configuration are also contemplated.

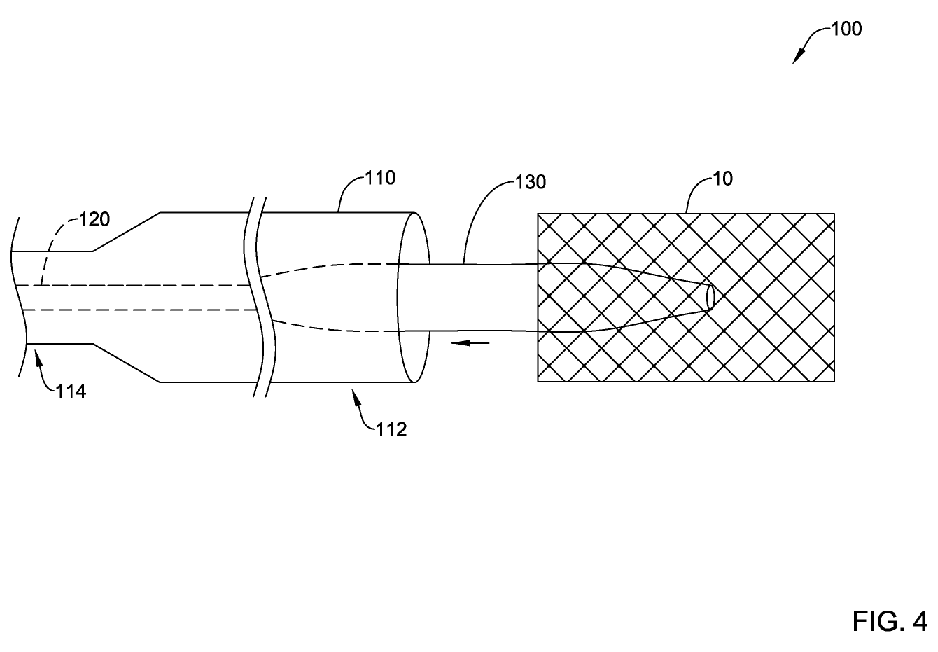

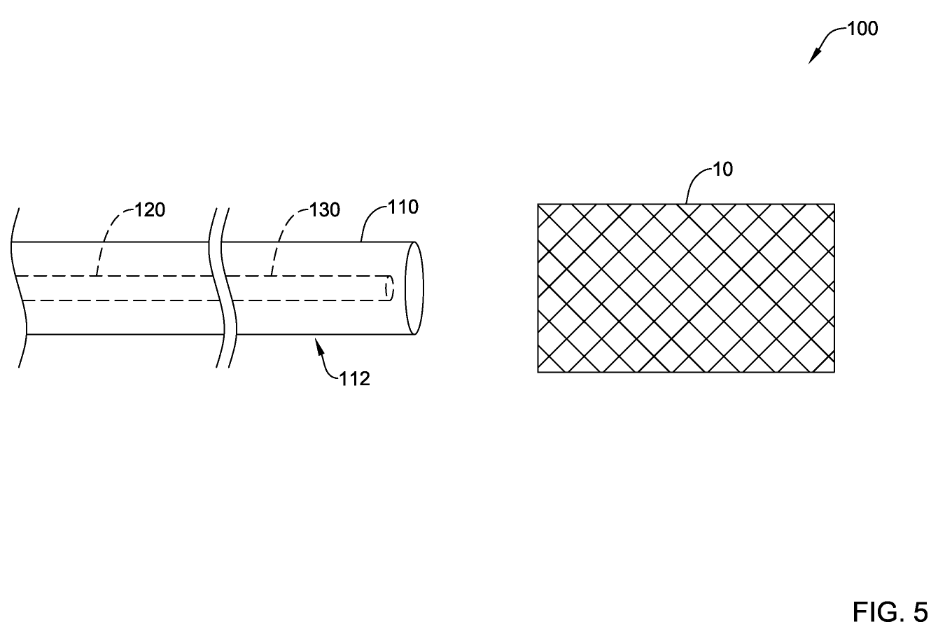

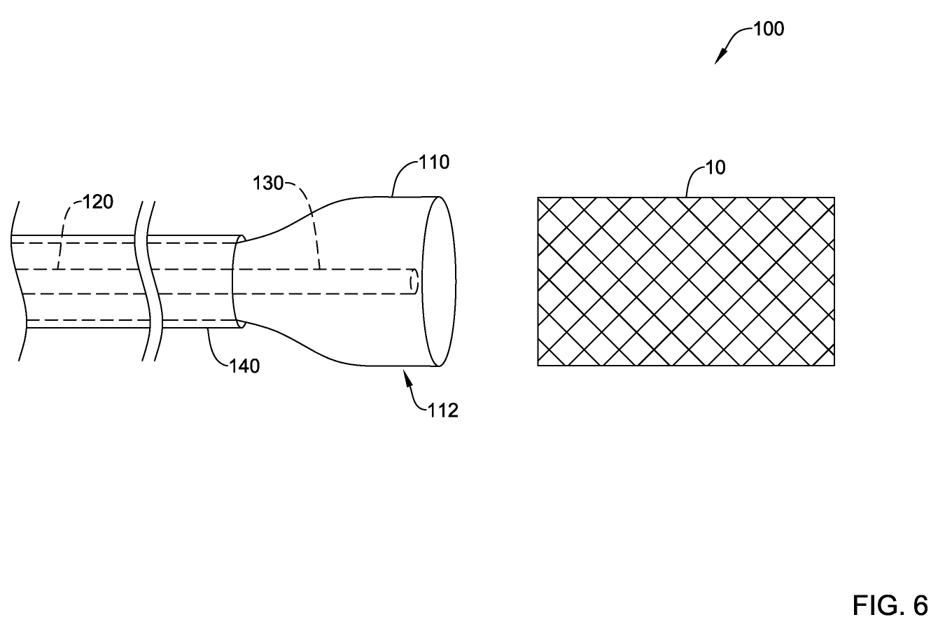

In some embodiments, the dilation balloon 130 may be configured to deflate toward and/or to the deflated configuration after translating and/or retracting the delivery sheath 110 relative to the elongate shaft 120 and/or the dilation balloon 130, and/or after deploying the self-expanding stent 10 at the target site, as seen in FIG. 4, to release the self-expanding stent 10 at the target site. In some embodiments, the distal expandable region 112 of the delivery sheath 110 may be configured to radially collapse toward the delivery configuration after deploying and/or releasing the self-expanding stent 10, as seen in FIGS. 5-6. In some embodiments, the distal expandable region 112 of the delivery sheath 110 may be self-biased toward the delivery configuration, as seen in FIG. 5. Accordingly, when a radially outward force applied to the distal expandable region 112 of the delivery sheath 110 by the dilation balloon 130 is removed, such as when or after retracting the delivery sheath 110 relative to the elongate shaft 120 and/or the dilation balloon 130, the distal expandable region 112 of the delivery sheath 110 may be configured to radially collapse toward the delivery configuration without any further intervention or application of force thereto.

In some embodiments, a guide sheath 140 may be advanced over the distal expandable region 112 of the delivery sheath 110 and/or the distal expandable region 112 of the delivery sheath 110 may be retracted into the guide sheath 140 to radially collapse the distal expandable region 112 of the delivery sheath 110 toward the delivery configuration after deploying and/or releasing the self-expanding stent 10, as seen in FIG. 6. Accordingly, when a radially outward force applied to the distal expandable region 112 of the delivery sheath 110 by the dilation balloon 130 is removed, such as when or after retracting the delivery sheath 110 relative to the elongate shaft 120 and/or the dilation balloon 130, the distal expandable region 112 of the delivery sheath 110 may be configured to retain its shape and thus will not radially collapse toward the delivery configuration without further intervention or application of force thereto. The guide sheath 140 may be configured to radially collapse and/or to apply a radially inward force to the distal expandable region 112 of the delivery sheath 110, thereby shifting the distal expandable region 112 of the delivery sheath 110 toward the delivery configuration. Other configurations are also contemplated.

In some embodiments, a method of delivering the self-expanding stent 10 may comprise positioning the stent delivery system 100 with the self-expanding stent 10 disposed therein in the collapsed configuration at a target site. As discussed herein, the stent delivery system 100 may comprise the delivery sheath 110 comprising the distal expandable region 112 and the proximal non-expandable region 114, and the elongate shaft 120 slidably disposed within the delivery sheath 110, wherein the elongate shaft 120 comprises the dilation balloon 130 at its distal end.

In some embodiments, the dilation balloon 130, in the deflated configuration, may be disposed within the self-expanding stent 10 in the radially collapsed configuration. In some embodiments, the distal expandable region 112 of the delivery sheath 110 may be configured to constrain the self-expanding stent 10 in the radially collapsed configuration. In some embodiments, the distal expandable region 112 of the delivery sheath 110 may be configured to constrain the self-expanding stent 10 in the radially collapsed configuration with the dilation balloon 130 disposed within the self-expanding stent 10 in the deflated configuration.

In some embodiments, the method of delivering the self-expanding stent 10 may comprise inflating the dilation balloon 130 to the inflated configuration within the distal expandable region 112 of the delivery sheath 110 at the target site with the self-expanding stent 10 disposed therein. In some embodiments, the method of delivering the self-expanding stent 10 may comprise inflating the dilation balloon 130 to the inflated configuration within the distal expandable region 112 of the delivery sheath 110 at the target site with the self-expanding stent 10 disposed within the distal expandable region 112 of the delivery sheath 110, wherein the dilation balloon 130 is disposed within the self-expanding stent 10 within the distal expandable region 112 of the delivery sheath 110 at the target site.

In some embodiments, inflating the dilation balloon 130 to the inflated configuration within the distal expandable region 112 of the delivery sheath 110 may shift the self-expanding stent 10 to the expanded configuration within the distal expandable region 112 of the delivery sheath 110 at the target site. In some embodiments, inflating the dilation balloon 130 to the inflated configuration within the distal expandable region 112 of the delivery sheath 110 may radially expand the distal expandable region 112 of the delivery sheath 110 toward and/or to the deployment configuration. In some embodiments, inflating the dilation balloon 130 to the inflated configuration within the distal expandable region 112 of the delivery sheath 110 in configured to simultaneously shift the self-expanding stent 10 to the expanded configuration within the distal expandable region 112 of the delivery sheath 110 and radially expand the distal expandable region 112 of the delivery sheath 110 toward and/or to the deployment configuration. Other configurations are also contemplated.

In some embodiments, the method of delivering the self-expanding stent 10 may comprise retracting the delivery sheath 110 relative to the elongate shaft 120 and/or the dilation balloon 130 with the dilation balloon 130 in the inflated configuration to move the self-expanding stent 10 out of the distal expandable region 112 of the delivery sheath 110 at the target site. In some embodiments, the method of delivering the self-expanding stent 10 may comprise retracting the delivery sheath 110 relative to the elongate shaft 120 and/or the dilation balloon 130 with the distal expandable region 112 of the delivery sheath 110 in the deployment configuration to move the self-expanding stent 10 out of the distal expandable region 112 of the delivery sheath 110 at the target site. In some embodiments, the method of delivering the self-expanding stent 10 may comprise, after inflating the dilation balloon 130 to the inflated configuration, retracting the delivery sheath 110 relative to the elongate shaft 120 and/or the dilation balloon 130 with the dilation balloon 130 in the inflated configuration to move the self-expanding stent 10 out of the distal expandable region 112 of the delivery sheath 110 at the target site. In some embodiments, the method of delivering the self-expanding stent 10 may comprise, after inflating the dilation balloon 130 to the inflated configuration, retracting the delivery sheath 110 relative to the elongate shaft 120 and/or the dilation balloon 130 with the distal expandable region 112 of the delivery sheath 110 in the deployment configuration to move the self-expanding stent 10 out of the distal expandable region 112 of the delivery sheath 110 at the target site. In some embodiments, in the inflated configuration the dilation balloon 130 may be configured to retain the self-expanding stent 10 thereon as the delivery sheath 110 is retracted relative to the elongate shaft 120 and/or the dilation balloon 130, as discussed herein.

In some embodiments, the method of delivering the self-expanding stent 10 may comprise, thereafter (e.g., after retracting the delivery sheath 110), deflating the dilation balloon 130 from the inflated configuration toward and/or to the deflated configuration to release the self-expanding stent 10 at the target site. In some embodiments, the method of delivering the self-expanding stent 10 may comprise radially collapsing the distal expandable region 112 of the delivery sheath 110 toward and/or to the delivery configuration, as described herein, and removing the stent delivery system 100 from the target site.

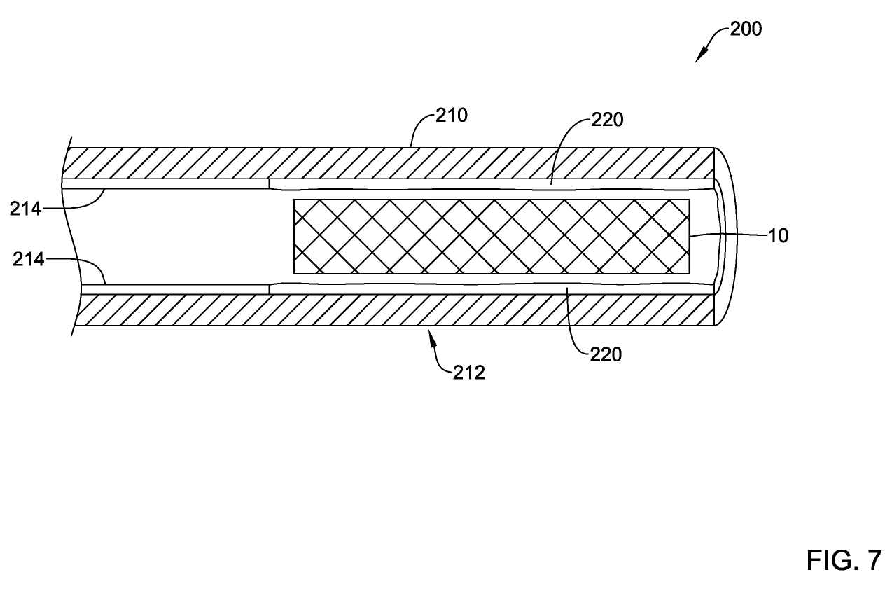

FIGS. 7-11 schematically illustrate selected aspects of a stent delivery system 200 and methods of delivering the self-expanding stent 10. The self-expanding stent 10 may be constructed and/or may comprise characteristics according to the instant disclosure. As discussed above, in some alternative embodiments, the stent may be a non-self-expanding stent.

In some embodiments, the stent delivery system 200 may comprise a delivery sheath 210 configured to receive a self-expanding stent 10 within a distal portion 212 of the delivery sheath 210 in a radially collapsed configuration. The delivery sheath 210 may comprise a lumen extending therein and/or proximally from a distal end of the delivery sheath 210. The delivery sheath 210 and/or the lumen thereof may be configured to receive the self-expanding stent 10 therein in the radially collapsed configuration.

The stent delivery system 200 may comprise a balloon 220 disposed within the distal portion 212 of the delivery sheath 210. In some embodiments, the balloon 220 may be disposed along an inner surface of the delivery sheath 210. In some embodiments, the balloon 220 may extend radially inward from the delivery sheath 210. In some embodiments, the balloon 220 may be fixedly attached to the inner surface of the delivery sheath 210. In some embodiments, the balloon 220 may be monolithically formed with the delivery sheath 210. Other configurations are also contemplated.

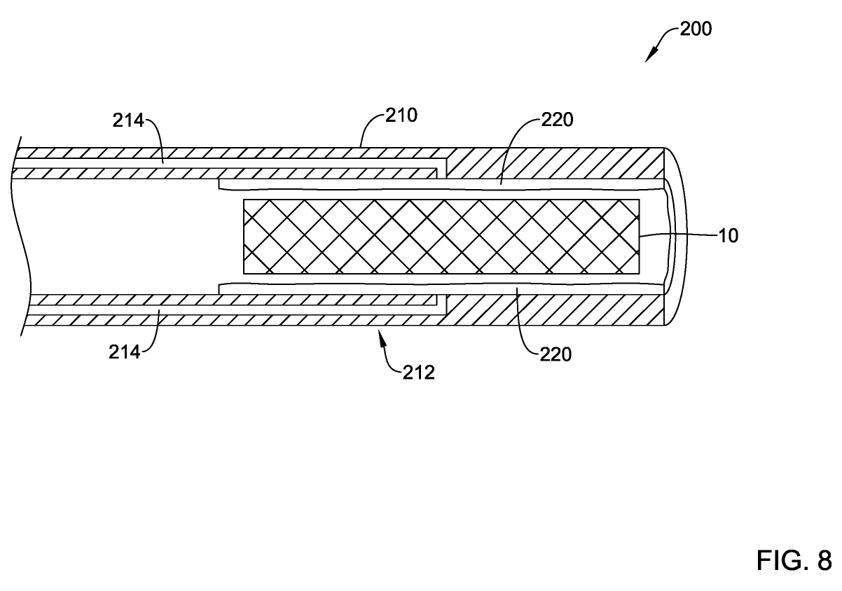

In some embodiments, the balloon 220 may be configured to shift between a deflated configuration (e.g., FIGS. 7-8) and an inflated configuration. In some embodiments, the delivery sheath 210 may be configured to receive the self-expanding stent 10 in the radially collapsed configuration when the balloon 220 is in the deflated configuration. In some embodiments, the self-expanding stent 10 in the radially collapsed configuration may be insertable into the distal portion 212 of the delivery sheath 210 when the balloon 220 is in the deflated configuration. Other configurations are also contemplated.

In some embodiments, the delivery sheath 210 may comprise at least one inflation lumen 214 in fluid communication with the balloon 220. The at least one inflation lumen 214 may be in fluid communication with a source of inflation media (e.g., fluid, gas, etc.). In some embodiments, the at least one inflation lumen 214 may be at least partially disposed along the inner surface of the delivery sheath 210, as seen in FIG. 7. In some embodiments, the at least one inflation lumen 214 may be at least partially disposed within the lumen of the delivery sheath 210. In some embodiments, the at least one inflation lumen 214 may be at least partially disposed within a side wall of the delivery sheath 210, as seen in FIG. 8. In some embodiments, the at least one inflation lumen 214 may be at least partially disposed along an outer surface of the delivery sheath 210. Other configurations, including combinations thereof, are also contemplated.

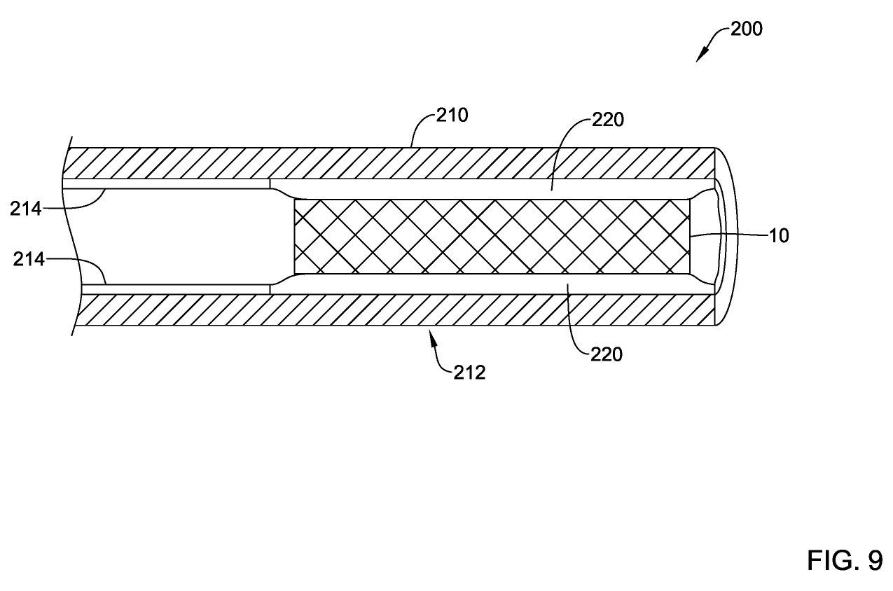

In some embodiments, the balloon 220 may be configured to constrain the self-expanding stent 10 in the radially collapsed configuration within the distal portion 212 of the delivery sheath 210, as seen in FIG. 9. In some embodiments, after the self-expanding stent 10 is disposed within the distal portion 212 of the delivery sheath 210 in the radially collapsed configuration, the balloon 220 may be partially inflated until the balloon 220 is engaged with an outer surface of the self-expanding stent 10. In a partially inflated configuration, the balloon 220 may be configured to prevent self-expansion of the self-expanding stent 10. With the balloon 220 in the partially inflated configuration, the delivery sheath 210 may be advanceable to the target site while holding the self-expanding stent 10 in the radially compressed configuration. When the self-expanding stent 10 is properly positioned within the distal portion 212 of the delivery sheath 210, a proximal portion 222 of the balloon 220 extends proximally of the self-expanding stent 10.

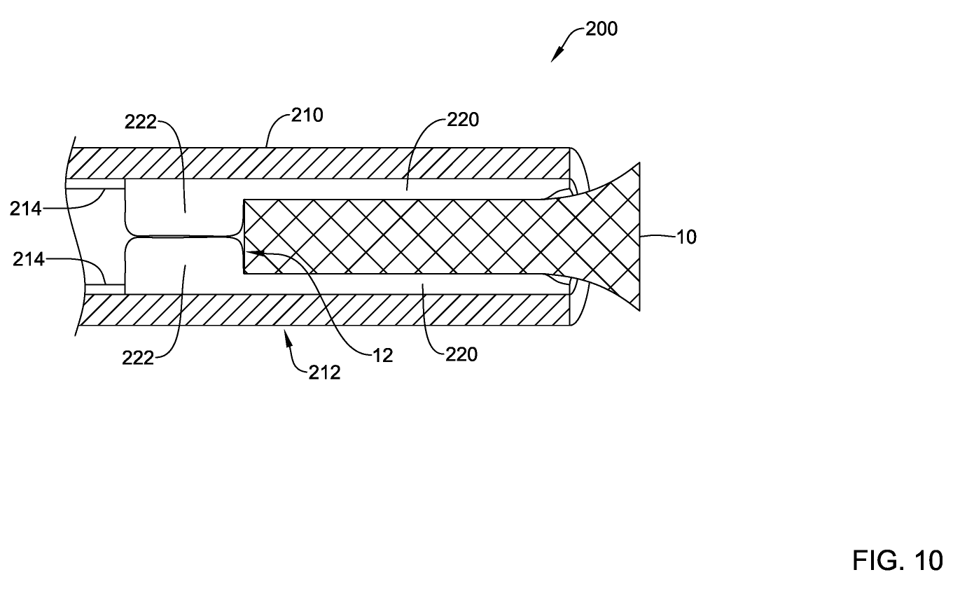

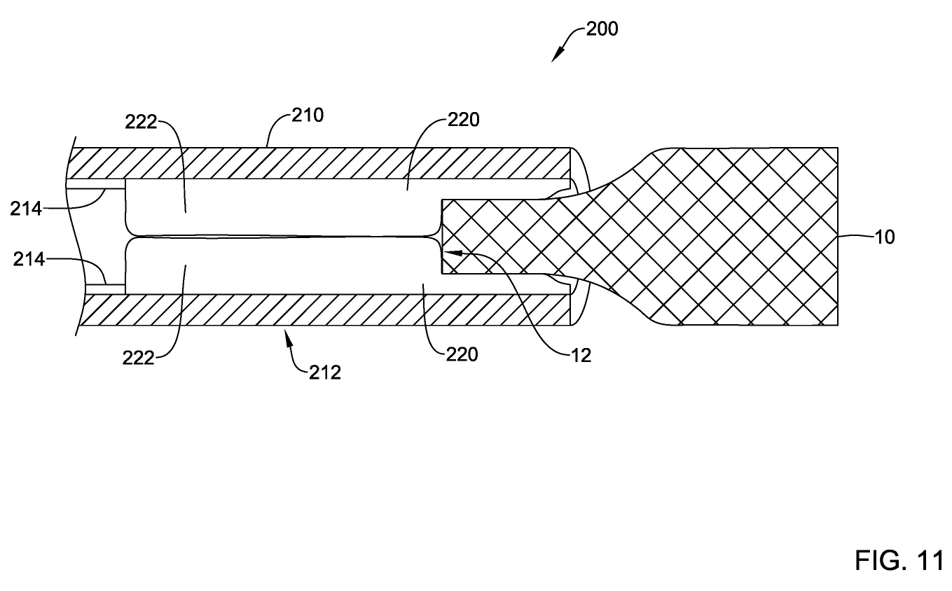

In some embodiments, after positioning the distal portion 212 of the delivery sheath 210 at the target site, the self-expanding stent 10 may be deployable from the distal portion 212 of the delivery sheath 210 at the target site. In at least some embodiments, the balloon 220 may be configured to advance the self-expanding stent 10 out of the distal portion 212 of the delivery sheath 210. In some embodiments, inflating the balloon 220 toward and/or to the inflated configuration may be configured to advance the self-expanding stent 10 out of the distal portion 212 of the delivery sheath 210. In some embodiments, as the balloon 220 inflates, the balloon 220 may be configured to expand radially inward from the delivery sheath 210 to advance the self-expanding stent 10 out of the distal portion 212 of the delivery sheath 210. The balloon 220 may be adapted and configured to expand radially inward in a directional manner or in a sequential manner as the balloon 220 inflates. For example, the proximal portion 222 of the balloon 220 extending and/or disposed proximal of the self-expanding stent 10 may expand radially inward and/or may be inflated first to engage a proximal end 12 of the self-expanding stent 10, as seen in FIG. 10. Thereafter, the balloon 220 may expand and/or inflate radially inward in a distal direction to gradually push the self-expanding stent 10 out of the distal portion 212 of the delivery sheath 210, as seen in FIG. 11.

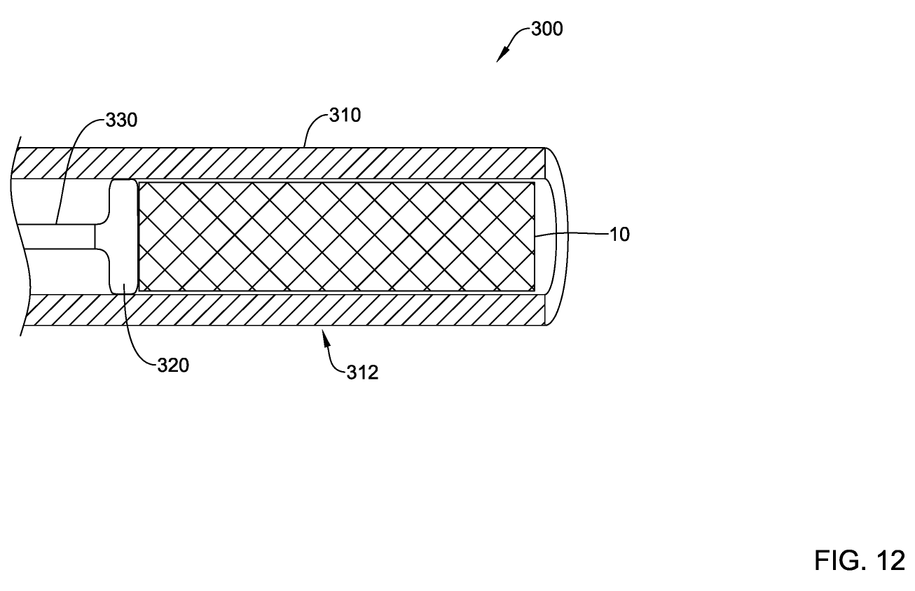

FIGS. 12-14 schematically illustrate selected aspects of a stent delivery system 300 and methods of delivering the self-expanding stent 10. The self-expanding stent 10 may be constructed and/or may comprise characteristics according to the instant disclosure. As discussed above, in some alternative embodiments, the stent may be a non-self-expanding stent.

In some embodiments, the stent delivery system 300 may comprise a delivery sheath 310 configured to receive a self-expanding stent 10 within a distal portion 312 of the delivery sheath 310 in a radially collapsed configuration. The delivery sheath 310 may comprise a lumen extending therein and/or proximally from a distal end of the delivery sheath 310. The delivery sheath 310 and/or the lumen thereof may be configured to receive the self-expanding stent 10 therein in the radially collapsed configuration.

The stent delivery system 300 may comprise a balloon 320 disposed within the distal portion 312 of the delivery sheath 310. In some embodiments, the balloon 320 may be fixedly attached to a distal end of an elongate shaft 330 disposed within the delivery sheath 310. In some embodiments, the balloon 320 may be monolithically formed with the elongate shaft 330. Other configurations are also contemplated.

In some embodiments, the balloon 320 may be configured to shift between a deflated configuration and an inflated configuration. In some embodiments, the delivery sheath 310 may be configured to receive the self-expanding stent 10 in the radially collapsed configuration when the balloon 320 is in the deflated configuration, as seen in FIG. 12. In some embodiments, the self-expanding stent 10 in the radially collapsed configuration may be insertable into the distal portion 312 of the delivery sheath 310 when the balloon 320 is in the deflated configuration. In some embodiments, the self-expanding stent 10 in the radially collapsed configuration may be insertable into the distal portion 312 of the delivery sheath 310 when the balloon 320 is in the inflated configuration. Other configurations are also contemplated.

In some embodiments, after positioning the distal portion 312 of the delivery sheath 310 at the target site, the self-expanding stent 10 may be deployable from the distal portion 312 of the delivery sheath 310 at the target site. In at least some embodiments, the balloon 320 may be configured to advance the self-expanding stent 10 out of the distal portion 312 of the delivery sheath 310.

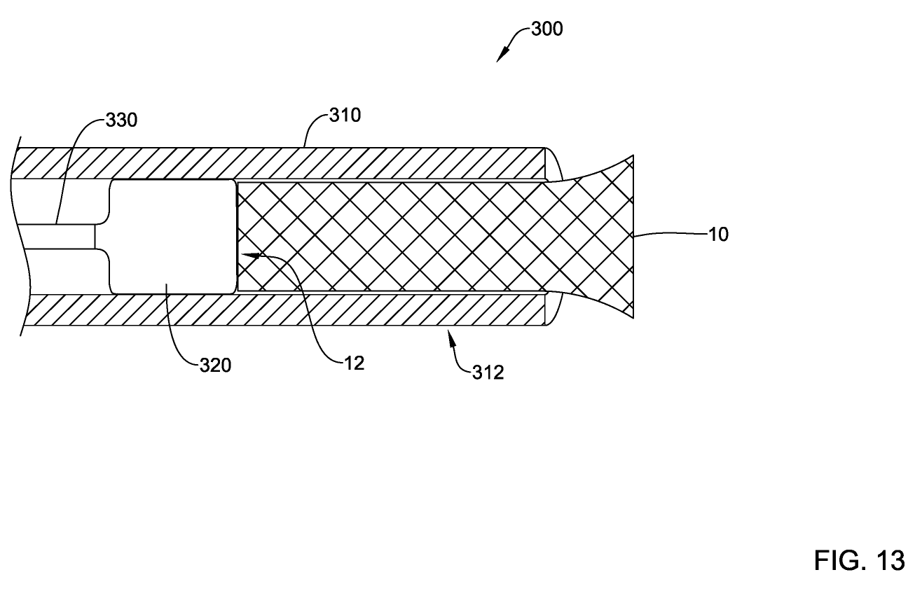

In some embodiments, inflating the balloon 320 toward and/or to the inflated configuration may be configured to advance the self-expanding stent 10 out of the distal portion 312 of the delivery sheath 310, as seen in FIG. 13. In some embodiments, the balloon 320 may be configured to expand and/or inflate distally from the distal end of the elongate shaft 330 to engage the proximal end 12 of the self-expanding stent 10, thereby advancing the self-expanding stent 10 out of the distal portion 312 of the delivery sheath 310 as the balloon 320 inflates.

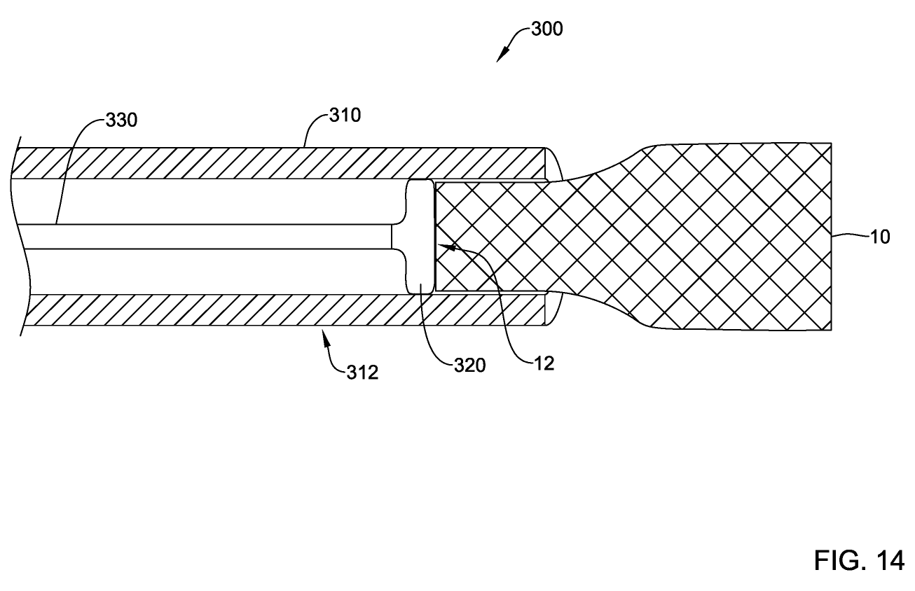

In some embodiments, the elongate shaft 330 and/or the balloon 320 is configured to advance distally within and/or relative to the delivery sheath 310 with the balloon 320 in the inflated configuration to push the self-expanding stent 10 out of the distal portion 312 of the delivery sheath 310, as seen in FIG. 14. In some embodiments, the elongate shaft 330 and/or the balloon 320 may be advanced distally within and/or relative to the delivery sheath 310 with the balloon 320 in the inflated configuration to engage the proximal end 12 of the self-expanding stent 10, thereby pushing the self-expanding stent 10 out of the distal portion 312 of the delivery sheath 310 as the balloon 320 is advanced distally within and/or relative to the delivery sheath 310.

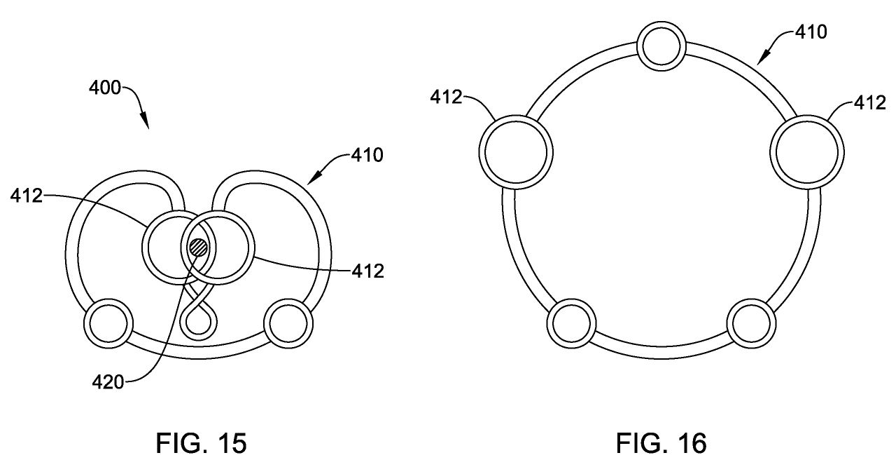

FIGS. 15-16 are end views schematically illustrating selected aspects of a self-expanding stent 410 and a stent delivery system 400. The self-expanding stent 410 may be constructed and/or may comprise characteristics according to the instant disclosure. As discussed above, in some embodiments, the stent may be a non-self-expanding stent.

In some embodiments, the stent delivery system 400 may comprise a delivery sheath (not shown) configured to receive the self-expanding stent 410 within a distal portion of the delivery sheath in a radially collapsed configuration. The delivery sheath may comprise a lumen extending therein and/or proximally from a distal end of the delivery sheath. The delivery sheath and/or the lumen thereof may be configured to receive the self-expanding stent 410 therein in the radially collapsed configuration.

The stent delivery system 400 may comprise a stent retention wire 420 slidably disposed within the delivery sheath. The stent retention wire 420 may be configured to engage with the self-expanding stent 410 in the radially collapsed configuration, as seen in FIG. 15. In some embodiments, the self-expanding stent 410 may comprise a plurality of retention loops 412 configured to receive the stent retention wire 420 therein in the radially collapsed configuration. In some embodiments, the plurality of retention loops 412 may comprise two retention loops. In some embodiments, the plurality of retention loops 412 may comprise three or more retention loops. Other configurations are also contemplated.

In some embodiments, the self-expanding stent 410 may be shifted into the radially collapsed configuration before self-expanding stent 410 is loaded into the distal portion of the delivery sheath. In the radially collapsed configuration, the plurality of retention loops 412 may be axially aligned with each other. After shifting the self-expanding stent 410 to the radially collapsed configuration (e.g., FIG. 15), the stent retention wire 420 may be positioned through the plurality of retention loops 412 to constrain the self-expanding stent 410 in the radially collapsed configuration. Thereafter, the self-expanding stent 410 may be loaded into the distal portion of the delivery sheath and advanced to the target site.

After positioning the delivery sheath and/or the self-expanding stent 410 at the target site, the self-expanding stent 410 may be deployed out of the distal portion of the delivery sheath at the target site. In some embodiments, the stent retention wire 420 may be translated proximally relative to the self-expanding stent 410 and/or the plurality of retention loops 412 to remove the stent retention wire 420 from the plurality of retention loops 412. Upon removing the stent retention wire 420 from the plurality of retention loops 412, the self-expanding stent 410 be configured to shift toward and/or to the radially expanded configuration, as seen in FIG. 16. In some embodiments, the self-expanding stent 410 may be deployed out of the distal portion of the delivery sheath at the target site before removing the stent retention wire 420 from the plurality of retention loops 412. In some alternative embodiments, the self-expanding stent 410 may be deployed out of the distal portion of the delivery sheath at the target site after removing the stent retention wire 420 from the plurality of retention loops 412 (e.g., the stent retention wire 420 may be removed from the plurality of retention loops 412 while the self-expanding stent 410 is still disposed within the distal portion of the delivery sheath, and the self-expanding stent 410 may thereafter be deployed and/or pushed out of the distal portion of the delivery sheath at the target site. Other configurations are also contemplated.

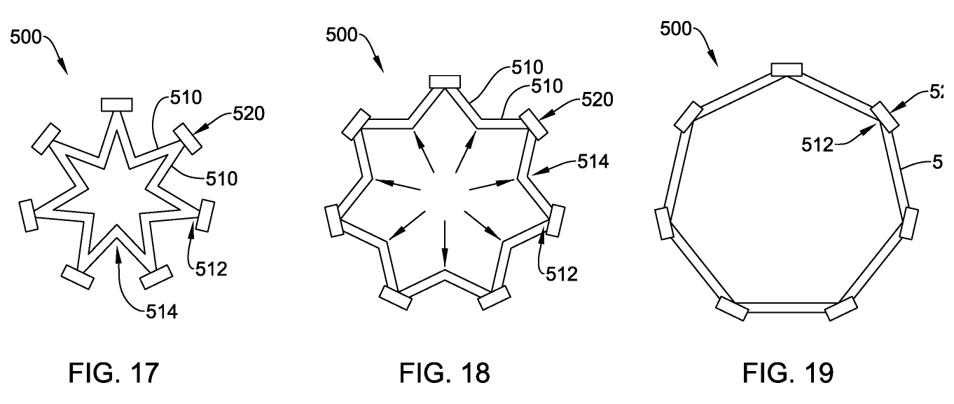

FIGS. 17-19 are end views schematically illustrating selected aspects of a stent 500 and methods of deploying the stent 500. The stent 500 may be constructed and/or may comprise characteristics according to the instant disclosure.

The stent 500 may be configured to shift between a radially collapsed configuration (e.g., FIG. 17) and a radially expanded configuration (e.g., FIG. 19). In the radially collapsed configuration, the stent 500 may comprise and/or may form a star-shaped cross-section comprising a plurality of undulating segments 510 extending between peaks 512 and valleys 514, as seen in FIG. 17. The plurality of undulating segments 510 may permit the stent 500 to fold upon itself when shifting into the radially collapsed configuration. In some embodiments, collapsing properties of the stent 500 may be configured to conform to the shape and/or size of a stent delivery system (e.g., a delivery sheath). In some embodiments, the stent 500 may comprise a plurality of locking elements 520 disposed at and/or configured to engage with the peaks 512 of the plurality of undulating segments 510.

Upon deploying the stent 500 at the target site (e.g., using a stent delivery system according to the disclosure, or using a stent delivery system known in the art), the stent 500 may be shifted from the radially collapsed configuration toward the radially expanded configuration, as seen in FIG. 18. In some embodiments, the stent 500 may be shifted from the radially collapsed configuration toward the radially expanded configuration using a dilation balloon. Other configurations are also contemplated.

After shifting the stent 500 to the radially expanded configuration, the plurality of locking elements 520 may be configured to engage with the plurality of undulating segments 510 at the peaks 512 to lock the stent 500 in the radially expanded configuration, as seen in FIG. 19, thereby preventing radially collapse of the stent 500 back toward the radially collapsed configuration. In some embodiments, the plurality of locking elements 520 may be configured to be selectively disengaged from the plurality of undulating segments 510 at the peaks 512 to unlock the stent 500 from the radially expanded configuration, such as for recapture and/or removal. Other configurations are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the stent, the stent delivery system, the delivery sheath, the elongate shaft, the balloon, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A stent delivery system, comprising:
a delivery sheath comprising a distal expandable region and a proximal non-expandable region; and
an elongate shaft slidably disposed within the delivery sheath, wherein the elongate shaft comprises a dilation balloon at its distal end;
wherein the distal expandable region is configured to constrain a self-expanding stent in a radially collapsed configuration with the dilation balloon disposed within the self-expanding stent in a deflated configuration;
wherein the dilation balloon is configured to inflate to an inflated configuration within the distal expandable region to shift the self-expanding stent to a radially expanded configuration within the distal expandable region.

2. The stent delivery system of claim 1, wherein the distal expandable region of the delivery sheath is configured to expand radially outward to a deployment configuration upon inflation of the dilation balloon to the inflated configuration within the distal expandable region.

3. The stent delivery system of claim 1, wherein the delivery sheath is retractable relative to the elongate shaft while the self-expanding stent is in the radially expanded configuration to deploy the self-expanding stent.

4. The stent delivery system of claim 3, wherein the dilation balloon is configured to retain the self-expanding stent thereon in the inflated configuration as the delivery sheath is retracted relative to the elongate shaft.

5. The stent delivery system of claim 3, wherein the distal expandable region is configured to radially collapse toward a delivery configuration after deploying the self-expanding stent.

6. The stent delivery system of claim 5, wherein the distal expandable region is self-biased toward the delivery configuration.

7. A method of delivering a self-expanding stent, comprising:
positioning a stent delivery system with the self-expanding stent disposed therein in a radially collapsed configuration at a target site, wherein the stent delivery system comprises:
a delivery sheath comprising a distal expandable region and a proximal non-expandable region; and
an elongate shaft slidably disposed within the delivery sheath, wherein the elongate shaft comprises a dilation balloon at its distal end;
inflating the dilation balloon to an inflated configuration within the distal expandable region of the delivery sheath with the self-expanding stent disposed within the distal expandable region of the delivery sheath;
retracting the delivery sheath relative to the elongate shaft with the dilation balloon in the inflated configuration to move the self-expanding stent out of the distal expandable region; and
thereafter, deflating the dilation balloon to release the self-expanding stent at the target site.

8. The method of claim 7, wherein the distal expandable region is configured to constrain the self-expanding stent in the radially collapsed configuration with the dilation balloon disposed within the self-expanding stent in a deflated configuration.

9. The method of claim 7, wherein inflating the dilation balloon to an inflated configuration within the distal expandable region of the delivery sheath shifts the self-expanding stent to a radially expanded configuration within the distal expandable region.

10. The method of claim 7, wherein inflating the dilation balloon to an inflated configuration within the distal expandable region of the delivery sheath radially expands the distal expandable region of the delivery sheath to a deployment configuration.

11. The method of claim 7, wherein in the inflated configuration the dilation balloon is configured to retain the self-expanding stent thereon as the delivery sheath is retracted.

12. The method of claim 7, further comprising radially collapsing the distal expandable region of the delivery sheath toward a delivery configuration and removing the stent delivery system from the target site.

13. The method of claim 12, wherein the distal expandable region of the delivery sheath is self-biased toward the delivery configuration.

14. A stent delivery system, comprising:
a delivery sheath configured to receive a stent within a distal portion of the delivery sheath in a radially collapsed configuration; and
a balloon disposed within the distal portion of the delivery sheath;
wherein the balloon is configured to advance the stent out of the distal portion of the delivery sheath.

15. The stent delivery system of claim 14, wherein the balloon is fixedly attached to the delivery sheath.

16. The stent delivery system of claim 15, wherein the balloon is configured to expand radially inward from the delivery sheath to advance the stent out of the distal portion of the delivery sheath.

17. The stent delivery system of claim 15, wherein the balloon is configured to constrain the stent in the radially collapsed configuration within the distal portion of the delivery sheath.

18. The stent delivery system of claim 14, wherein the balloon is fixedly attached to a distal end of an elongate shaft disposed within the delivery sheath.

19. The stent delivery system of claim 18, wherein the balloon is configured to expand distally from the distal end of the elongate shaft to advance the stent out of the distal portion of the delivery sheath.

20. The stent delivery system of claim 18, wherein the elongate shaft is configured to advance distally relative to the delivery sheath with the balloon in an inflated configuration to push the stent out of the distal portion of the delivery sheath.
